(19) United States
(12) Patent Application Publication
(10) Pub. No.: US 2003/0023157 A1
(43) Pub. Date: Jan. 30, 2003

(54) Method for the operation of a magnetic resonance apparatus

(76) Inventor: Matthias Gebhardt, Erlangen (DE)

Correspondence Address: SCHIFF HARDIN & WAITE, 6600 SEARS TOWER, 233 S WACKER DR, CHICAGO, IL, 60606-6473, US

(21) Appl. No.: 10/188,730

(22) Filed: Jul. 2, 2002

(30) Foreign Application Priority Data
Jul. 5, 2001 (DE) 10132593.2

(51) Int. Cl. A61B 5/05

(52) U.S. Cl. 600/410

(57) ABSTRACT

In a method for the operation of a magnetic resonance apparatus, eddy current fields that are caused by at least one magnetic gradient field are at least partially compensated by means of a compensation setting, and the compensation setting is adapted dependent on a position of a mid-point of a region of an examination subject to be imaged with respect to a mid-point of an imaging volume of the magnetic resonance apparatus.

### BACKGROUND OF THE INVENTION

1. Field of the Invention

The present invention is directed to a method for the operation of a magnetic resonance apparatus, whereby eddy current fields that are caused by at least one magnetic gradient field are at least partially compensated by means of a compensation setting.

2. Description of the Prior Art

Magnetic resonance technology is a known technique for acquiring images of the inside of the body of an examination subject. To that end, rapidly switched gradient fields that are generated by a gradient coil system are superimposed on a static basic magnetic field generated by a basic field magnetic in a magnetic resonance apparatus. Further, the magnetic resonance apparatus has a radio-frequency system that emits radio-frequency signals into the examination subject for triggering magnetic resonance signals and that picks up the generated magnetic resonance signals, from which magnetic resonance images are generated.

A gradient coil of the gradient coil system generates a gradient field for a specific spatial direction. Ideally, it is desirable for the gradient field to have only a field component that is co-linear with the basic magnetic field, at least within the imaging volume of the magnetic resonance apparatus. The field component has a prescribable gradient that is of approximately the same magnitude at any arbitrary point in time, at least within the imaging volume, independent of location. Since the gradient field is a time-variable magnetic field, the above applies to every point in time; from one point in time to another point in time, however, the strength of the gradient varies. The direction of the gradient is usually permanently prescribed by the design of the gradient coil. Appropriate currents are set in the gradient coil for generating the gradient field. The amplitudes of the required currents amount to several 100 A. The current rise and decay rates amount to several 100 kA/s. The gradient coils are connected to gradient amplifiers for the power supply.

It is known, for example from German OS 199 55 117, to describe a magnetic flux density B(r, θ, φ) of a gradient field for a prescribable intensity of current by means of a spherical function development according to the following equations:

$$B ( r , θ , ϕ ) = {\sum }_{k = 0}^{∞} {\sum }_{m = - k}^{+ k} {A}_{( k , m )} \cdot {r}^{k} \cdot {Y}_{( k , m )} ( θ , ϕ ) with$$

$${Y}_{( k , m )} ( θ , ϕ ) = \{ \begin{matrix}{P}_{( k , m )} ( cos θ ) \cdot cos ( m ϕ ) & m = 0 , 1 , 2 , … , k \\ {P}_{( k , \vert m \vert} ( cos θ ) \cdot sin ( \vert m \vert ϕ ) & m = - 1 , - 2 , … , - k\end{matrix}$$

wherein A_{(k,m)} are the spherical coefficients with a suitable normalization. The radius r as well as the angles θ and φ—as spherical coordinates—describe a point of the three-dimensional space proceeding from an origin. The origin is generally defined in the center of the gradient coil system. P_{(k,m)} (cos θ) and P_{(k,|m|)} (cos θ) are allocated Legendre polynomials or functions dependent on cos θ. The value for k indicates the order of a field component of the gradient field belonging to the respective coefficient A_{(k,m)}. A field component of the 0^{th} order means a field component similar to a basic magnetic field. An ideal gradient field has only a field component of the first order, i.e. all coefficients A_{(k,m)} other than the coefficient A_{(1,0)} or A_{(1,1)} are equal to zero. Real gradient fields usually exhibit disturbances in the form of field components, particularly of a higher order having an odd-numbered k.

The gradient coil system usually is surrounded by conductive structures wherein eddy currents are induced by the activated gradient fields. Examples of such conductor structures are a vacuum container and/or a cryoshield of a superconducting basic field magnet, a copper foil of a radio-frequency shielding and the gradient coil itself. The eddy current fields generated by the eddy currents are unwanted because they attenuate the gradient fields and distort them in terms of their time curve, unless counter-measures are undertaken. This leads to degradation of the quality of the magnetic resonance images. This also applies to an actively shielded gradient coil system that has shielding coils belonging to the gradient coils, although a quantitative reduction of the eddy currents is achieved compared to the unshielded gradient coil system.

The distortion of a gradient field as a consequence of the eddy current fields can be compensated by a suitable pre-distortion of a quantity controlling the gradient field, to a certain extent. For compensation, the controlling quantity is filtered such that the eddy current fields that occur given operation of the gradient coil without the pre-distortion are canceled by the pre-distortion. Similar to that described above for gradient fields, the eddy current fields can be described in the form of a spherical function development. For this purpose a temporally decreasing exponential function characterized by a time constant is allocated to each coefficient for describing the time-dependency of the eddy current field components. A filter network can be utilized for this filtering, having dimensions defined by the time constants and coefficients that can be determined, for example, with a method disclosed in German PS 198 59 501.

At least cross-terms of an eddy current field are identified with the method for acquiring eddy currents disclosed in German PS 198 59 501. One cross-term is a field component of the eddy current field that is produced by the gradient field having a gradient in a first direction, with the field component that is effective in a second direction that is perpendicular to the first. When, for example, the field component is a field component of the first order, then the field component can be compensated by a suitable, oppositely directed drive of a gradient coil, with which a gradient field having a gradient in the second direction can be generated. For this purpose, a spatially expanded phantom is introduced into the imaging volume of the magnetic resonance apparatus; a test gradient pulse having a prescribable pulse width is activated and, after the deactivation of the test gradient pulse, at least two imaging sequence blocks that are spaced from one another in time are generated, and as at least two-dimensional dataset is produced from their imaging magnetic resonance signals. The phase information contained in the magnetic resonance signals contains characteristic quantities of the eddy current field. The aforementioned amplitudes and time constants of eddy currents can be determined therefrom with a suitable analysis method.

Coils referred to as shim coils also are known for use in magnetic resonance apparatus, the basic magnetic field being able to be homogenized therewith, for example, dependent on different examination subjects. The shim coils are operated with suitable direct currents for this purpose. Since linear basic magnetic field deviations, i.e. disturbances of the first order, can be compensated by charging the gradient coils with a direct current, the shim coils are usually fashioned such that exactly each shim coil compensates one disturbance of a specific order higher than the first order. Said German PS 9859 501 discloses, additionally, that disturbances of a higher order caused by eddy currents can be compensated by means of an additional pulsed charging of the shim coils with currents.

For correcting a dynamic disturbance of the basic magnetic field, further, it is known to adapt the temporal sequence of a complete examination sequence to the dynamics of the disturbance.

Magnetic resonance images of good quality can be produced with the known compensation methods for a region to be imaged having a mid-point that coincides with the mid-point of the imaging volume of the magnetic resonance apparatus. If, however, these mid-points deviate from one another, high-quality magnetic resonance images can be generated only using sequences referred to as gradient-balanced sequences. Compared to non-gradient-balanced sequences, however, gradient-balanced sequences exhibit the disadvantage that their implementation consumes a longer time duration. For regions to be imaged that come to lie exclusively in the edge regions of the imaging volume due to the design quality of the magnetic resonance apparatus and the physiognomy of the examination subject, quality problems can occur given employment of a non-gradient-balanced sequence. A typical example is shoulder imaging of a patient in a magnetic resonance apparatus having a tunnel-like examination space. The quality problems include unwanted distortions, an incomplete fat-water separation and/or fat saturation problems.

### SUMMARY OF THE INVENTION

An object of the present invention is to provide an improved method of the type initially described with which, among other things, a high-quality magnetic resonance image of a region to be imaged having a mid-point spaced from the mid-point of the imaging volume can be generated with a fast sequence.

The above object is achieved in accordance with the principles of the present invention in a method for operating a magnetic resonance apparatus, wherein eddy current fields exist that are caused by at least one magnetic gradient field, and wherein the eddy current fields are compensated using a compensation setting, and wherein the compensation setting is adapted dependent on the position of the mid-point of a region of the examination subject to be imaged, relative to the mid-point of an imaging volume of the magnetic resonance apparatus.

The invention is based on the perception that, in the known methods, a compensation of dynamic field disturbances that, corresponding to a spherical function development, exhibit higher orders around the mid-point of the imaging volume is deficient in edge regions of the imaging volume. According to the invention, a compensation adapted to the position of the imaged region within the imaging volume is implemented for a region to be imaged wherein the mid-point is spaced from a mid-point of the imaging volume. The eddy current-caused field disturbances thus can be presented in the form of a spherical function development around the mid-point of the region to be imaged. Compared to a spherical function development around the mid-point of the imaging volume, the spherical development around the mid-point of the region to be imaged better approximates reality in the imaged region, so that disturbances that remain uncompensated as disturbances of a higher order given a spherical function development around the mid-point of the imaging volume also are able to be compensated. It is advantageous that suppression of cost-terms for correcting gradient filed-like disturbances for each gradient axis, and a correction of dynamic disturbances similar to the basic magnetic field, already can be implemented given the initially described eddy current compensation methods of the prior art.

As a modification of the method disclosed by German PS 198 59 501, for example, the inventive method can be implemented during an initialization of the magnetic resonance apparatus by the phantom being additionally centered in relevant edge regions of the imaging volume and compensation settings optimized for the relevant edge regions being determined with a slice excitation that is correspondingly offset into the edge region. Dependent on a position of the region to be imaged within the imaging volume, the corresponding compensation setting values are then employed, or are interpolated between compensation setting values, during operation of the magnetic resonance apparatus.

The advantages that accompany a spherical function development around a different incident point are explained below with reference to a one-dimensional example and given the assumption that disturbances caused by eddy currents that, corresponding to a spherical function development around a prescribable incident point, exhibit orders greater than or equal to two cannot be compensated as a consequence of the system. According to a spherical function development around the mid-point of the imaging volume to which a zero point of a z-axis is allocated, an eddy current field has a disturbance A_{(3,0)} of the third order. The disturbance A_{(3,0)} exhibits a direct proportionality to the third power of the z-coordinate, i.e. it exhibits a direct proportionality to z₀³ at a point z₀ on the positive z-axis. According to the assumption, the disturbance A_{(3,0)} is not compensated. In a magnetic resonance imaging for a region to be imaged that has a mid-point that deviates from the zero point of the z-axis and, for example, is the point z₀, this means the magnetic resonance image has a distortion dependent on a quantity that the disturbance A_{(3,0)} exhibits around the point z₀.

The disturbance A_{(3,0)} is then locally observed in the environment around the point z₀. To that end, a further coordinate \check{z} is initially introduced, the zero point thereof corresponding to the point z₀. According to the spherical function development around the mid-point of the imaging volume, the disturbance A_{(3,0)} in the environment of the point z₀ thus is directly proportional to (z₀+\check{z})³. A presentation of (z₀+\check{z})³ as a series yields z₀³+3z₀²\check{z}+S (\check{z}²). S (\check{z}²) represents a residual disturbance that is clearly smaller, particularly for a region |\check{z}|<z₀, compared to z₀³.

Transferred to a spherical function development around the point z₀ and given the above assumption that only disturbances of the z^{th} and first order can be compensated, this means that, differing from the spherical function development around the mid-point of the imaging volume, the term z₀³ of the aforementioned series, as a disturbance of the 0^{th} order, as well as the term 3z₀²\check{z}, as disturbance of the first order, can be compensated. Only the residual disturbance S(\check{z}²) thus remains for the region to be imaged around the point zₒ, and this, as set forth above, is smaller than the disturbance A_{(3,o)} deriving from a spherical function development around the mid-point of the imaging volume that is directly proportional to zₒ³ at the point z₀. Thus, far fewer distortions occur for the region to be imaged around the point z₀ with a compensation of the eddy current field that is based on a spherical function development of the eddy current field around the point z₀ than given a compensation based on a spherical function development of the eddy current field around the mid-point of the imaging volume.

### DESCRIPTION OF THE DRAWINGS

FIG. 1 is a flowchart of the inventive method for the operation of a magnetic resonance apparatus having a temporally preceding initialization of the magnetic resonance apparatus.

FIG. 2 is a cross-section through a magnetic resonance apparatus having a tunnel-like examination space for explaining the inventive method.

### DESCRIPTION OF THE PREFERRED EMBODIMENTS

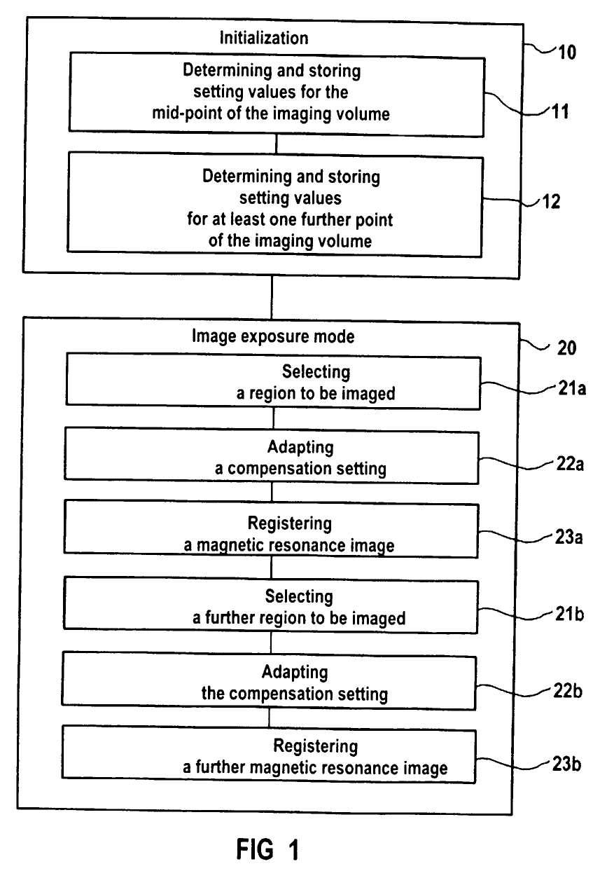

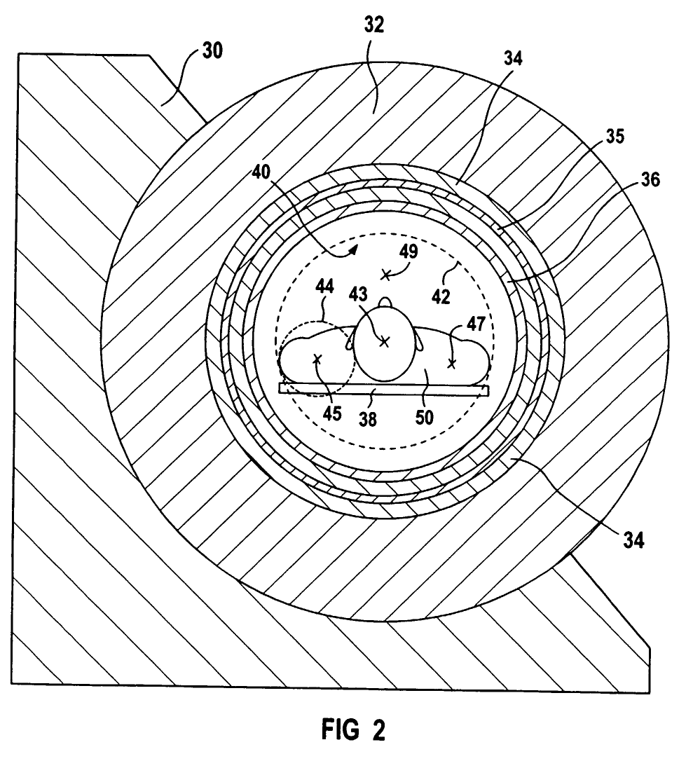

As an exemplary embodiment of the invention, FIG. 1 shows a flowchart of a method for the operation of a magnetic resonance apparatus having a temporally preceding initialization of the magnetic resonance apparatus. For assisting in the explanation of the flowchart of FIG. 1, the magnetic resonance apparatus 30 shown as an example in FIG. 2 is described in advance below.

FIG. 2 shows a cross-section through the magnetic resonance apparatus 30. The magnetic resonance apparatus 30 has an essentially hollow-cylindrical, superconducting basic field magnet 32 for generating a static basic magnetic field that is optimally uniform at least within a spherical imaging volume 42. Further, the magnetic resonance apparatus 30 has an essentially hollow-cylindrical gradient coil system 34 for generating rapidly switchable magnetic gradient fields that are optimally linear within the imaging volume 42. The gradient coil system 34 is arranged in the hollow of the basic field magnet 32. A shim coil system 35 with which, among other things, the basic magnetic field can be homogenized within the imaging volume 42 is integrated into the gradient coil system 34. Further, an antenna system 36 that is also essentially hollow-cylindrical is arranged in the interior of the gradient coil system 34, which emits radio-frequency signals for triggering magnetic resonance signals into an examination subject that is at least partially placed in the imaging volume 42, and which picks up the generated magnetic resonance signals. The antenna system 36 essentially forms a spatial limitation of a tunnel-like examination space 40. In order to introduce the examination subject, for example a patient 50, into the examination space 40 and in order to position a region of the patient 50 to be imaged in the imaging volume 42, the magnetic resonance apparatus 30 has a displaceable support mechanism 38 with which the patient 50 borne thereon can be introduced into the examination space 40 and with which the region to be imaged can be positioned in the imaging volume 42.

The flowchart of FIG. 1 is explained in greater detail below with reference to the magnetic resonance apparatus 30 FIG. 2. In a fist step 10 of the flowchart, an initialization of the magnetic resonance apparatus 30 is implemented after the magnetic resonance apparatus 30 is installed at its place of utilization. The initialization of the step 10 includes, among other things, the two steps 11 and 12 wherein setting values for the gradient and shim coil systems 34 and 35 are determined and stored in the magnetic resonance apparatus 30, so that unwanted influences of eddy current fields that are essentially produced by the activated gradient fields can be compensated in an image exposure mode in step 20. To that end, setting values that are optimized in view of a region to be imaged with a mid-point that is the same as a mid-point 43 of the imaging volume 42 are initially determined and stored in step 11 for this purpose. According to German PS 198 59 501 cited above (which corresponds to U.S. Pat. No. 6,335,620, the teachings of which are incorporated herein by reference), a spatially extensive phantom is centered in the imaging volume 42, at least one test gradient pulse having a prescribable pulse width is activated, at least two imaging sequence blocks spaced in time from one another are generated from a slice around the mid-point 43, and an at least two-dimensional dataset is generated from these imaging magnetic resonance signals. The setting values are thereby determined and stored as characteristic quantities of the eddy current fields that are encoded in the phase information contained in the magnetic resonance signals.

Setting values that are optimized for a region to be imaged having a mid-point that is spaced from the mid-point 43 of the imaging volume 42 are determined and stored in the further step 12. This region to be imaged can, for example, can be a region 44 to be imaged in the patient 50 around the shoulders of the patient 50 having a mid-point 45 as shown in FIG. 2. Due to the design nature of the magnetic resonance apparatus 30 and the physiognomy of the patient 50, it is particularly the region 44 of the shoulders of the patient 50 that are not centered in the imaging volume 42. The phantom is centered with respect to the mid-point 45 in Step 12 and the imaging magnetic resonance signal is derived from a slice around the mid-point 45, with the setting values optimized for the mid-point 45 being determined and stored according to the preceding step 11. In an analogous application of the step 12, further setting values that are optimized in view of the respective mid-point 45 can be determined and stored for further mid-points in the framework of the initialization of the step 10. Examples of further mid-points are a point 47 of the imaging volume 42 in the region of the other shoulder of the patient 50 and a point 49 of the imaging volume 42 that is arranged above the mid-point 43 in the illustrated cross-sectional area of the imaging volume 42 of FIG. 2.

After the initialization of Step 10 has ended, the magnetic resonance apparatus 30 is operated for registering magnetic resonance images in Step 20 of the flowchart. In step of 21a of the flowchart, a region to be imaged is to be selected for the patient 50, this region, for example, being the head of the patient 50. To that end, the patient 50—as shown in FIG. 2—is introduced into the examination space 40 lying down, so that the head of the patient 50 is centered in the imaging volume 42. Due to the nature of the magnetic resonance apparatus 30 as well as the physiognomy of the patient 50, it is thereby possible to position the patient 50 such that the mid-point of the region to be imaged, i.e. the mid-point of the head, is approximately identical to the mid-point 43 of the imaging volume 42. Before the exposure of magnetic resonance images of the head in Step 23a, an adaptation of a compensation setting ensues in Step 22a, causing an operation of the gradient coil system 34 during the magnetic resonance image exposure of Step 23a having a pre-distortion and/or causing a pulsed operation of the shim coil system 35. Since the mid-point of the head coincides with the mid-point 43 of the imaging volume 42, the setting values determined in Step 11 are selected in Step 22a as compensation setting and are correspondingly employed in the image exposure of the Step 23a.

The intent in Steps 21b through 23b of the flowchart is to likewise register optimally distortion-free magnetic resonance images for another region to be imaged. In Step 21b, for example, the shoulder region 44 of the patient 50 is selected as further region to be imaged. Due to the nature of the magnetic resonance apparatus 30 as well as the physiognomy of the patient 50, the shoulder region 44 of the patient cannot be positioned in the examination space 40 so that the mid-point 45 of the shoulder region 44 coincides with the mid-point 43 of the imaging volume 42. As shown in FIG. 2, the shoulder region 44 to be imaged extends into an edge region of the imaging volume 42. So that optimally distortion-free magnetic resonance images of the shoulder region 44 can be registered in the Step 23b with a non-gradient-balanced sequence, the compensation setting is adapted in a Step 22b. To that end, the setting values determined and stored in Step 12 for the mid-point 45 are selected and are correspondingly employed in the image exposure of Step 23b.

For further regions of the patient 50 to be imaged or of other examination subjects for whose mid-points no setting values had been determined and stored during the initialization of Step 10, those setting values for points of the imaging volume that lie optimally close to the respective mid-point of the region to be imaged are to be interpolated for the adaptation of the compensation setting, or setting values of that point that lies closest to the mid-point of the region to be imaged are selected.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for operating a magnetic resonance apparatus wherein eddy current fields exist that are caused by at least one magnetic gradient field, said magnetic resonance apparatus having an imaging volume with a mid-point, and having a plurality of components, said method comprising the steps of:
generating a compensation setting for at least one of said components to compensate said eddy current fields; and
adapting said compensation setting dependent on a mid-point of a region of an examination subject to be imaged relative to said mid-point of said imaging volume.

2. A method as claimed in claim 1 wherein said components include a gradient coil system, and comprising the step of using said compensation setting to modify operation of said gradient coil system.

3. A method as claimed in claim 1 wherein said components include a shim coil system, and comprising using said compensation setting for pulsed operation of said shim coil system.

4. A method as claimed in claim 1 wherein said components include a gradient coil system and a shim coil system, and comprising using said compensation setting to modify operation of said gradient coil system and for pulsed operation of said shim coil system.

5. A method as claimed in claim 1 wherein the step of generating said compensation setting comprises generating said compensation setting from a plurality of stored setting values.

6. A method as claimed in claim 5 comprising generating said setting values for said mid-point of said imaging volume and for a predetermined plurality of predetermined points in said imaging volume.

7. A method as claimed in claim 6 comprising generating said setting values during initialization of said magnetic resonance apparatus using a phantom disposed in said imaging volume.

8. A method as claimed in claim 7 comprising generating said setting values by obtaining magnetic resonance signals from said phantom in a layer proceeding substantially uniformly around each of said further points, and using phase information contained in said magnetic resonance signals to determine said setting values.

9. A method as claimed in claim 1 comprising generating said compensation setting by interpolation among a plurality of setting values.

10. A method as claimed in claim 9 comprising generating said setting values for said mid-point of said imaging volume and for a predetermined plurality of predetermined points in said imaging volume.

11. A method as claimed in claim 10 comprising generating said setting values during initialization of said magnetic resonance apparatus using a phantom disposed in said imaging volume.

12. A method as claimed in claim 11 comprising generating said setting values by obtaining magnetic resonance signals from said phantom in a layer proceeding substantially uniformly around each of said further points, and using phase information contained in said magnetic resonance signals to determine said setting values.
